(19) United States
(12) Patent Application Publication
(10) Pub. No.: US 2006/0229255 A1
(43) Pub. Date: Oct. 12, 2006

(54) Identification of n-alkylglycine trimers for induction of apoptosis

(76) Inventors: Gema Tarrason, Barcelona (ES); Claudia Cases, Barcelona (ES); Jaume Piulats, Barcelona (ES); Angel Messeguer, Barcelona (ES); Nuria Cortes, Sant Cugat del Valles (ES); Isabel Masip, Barcelona (ES)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/553,285

(22) PCT Filed: Apr. 8, 2004
(86) PCT No.: PCT/EP04/03749

§ 371 Date: Oct. 14, 2005

(30) Foreign Application Priority Data
Apr. 15, 2003 (EP) 03008604.5

(51) Int. Cl. A61K 38/04; A61K 31/337

(52) U.S. Cl. 514/19; 514/449

(57) ABSTRACT

N-alkylglycine trimers with the capacity to arrest the cell cycle in human cancer cells and to induce apoptosis useful for the therapy of cancer. Combination of said N-alkylglycine trimers with taxol.

### SUMMARY OF THE INVENTION

The inventions relates to the identification, synthesis and purification of two pseudopeptides herein named N10-13-10C and N13-13-10C derived from the screening of a library of trimers of N-alkylglycines. The compounds have the capacity to arrest the cell cycle followed by the induction of apoptosis in a human cancer cells.

### STATE OF THE ART

Cell proliferation is an ordered, tightly regulated process involving multiple checkpoints that integrate extra cellular growth signals, cell size, and DNA integrity. The somatic cell cycle is divided into an DNA synthesis phase (S phase) and a mitotic phase, in which a single cell divides into two daughter cells. These phases are separated by two gap phases (G1 and G2).

The vast majority of cells in the human body exist in a non-dividing, terminally differentiated state, the G0 phase. However, appropriate external stimuli, such as growth factors, cell-cell contact and adhesion to extra cellular matrix, regulate the catalytic activity of cyclin-dependent kinase (Cdks) and therefore the formation of replication origins. Phosphorylation of pRb by specific Cdks impairs binding to E2F/DP, allowing the progression from the G1 to the S phase (Chellappan, s. P., et al., 1991), and is negatively regulated by Cdk inhibitors, such as p15^{INK4b}, p16^{INK4a}, p21^{Cip1}, and p27^{KIP1} (Sherr, C. J. and Roberts, J. M., 1995). After successful completion of DNA synthesis, cells enter G2 phase in preparation for mitosis. Once started, DNA replication must be finished. The G1 restriction point divides the cell cycle into a growth factor dependent early G1 and a growth factor independent phases from late G1 through mitosis. Signaling pathways determine whether early G1 phase cells transit the restriction point to undergo eventual cellular division or, because of insufficient signaling strength, exit the cell cycle, and enter into G0, or enter in apoptosis. The overall balance of pro- and anti-apoptotic signals determines the fate of the cell.

Neoplastic cells acquire genetic alterations which disarrange homeostatic mechanisms that either minimize cells loss, i.e. suppress apoptosis, and/or enhance deregulated proliferation. A common feature of human cancer cells is inactivation of p16, over expression of Cyclin D and/or inactivation of pRb (Hall, M. and Peters, G., 1996). Induction of apoptosis in tumor cells and/or in non-tumor cells supporting tumor growth such as endothelial cells is a prime goal in cancer therapy. Cancer cells are usually more resistant to apoptosis due to mutations in some components of the apoptotic machinery.

Taxol is among the drugs with the broadest antineoplastic spectrum presently used in oncology. Taxol stabilizes microtubules and inhibits depolymerization back to tubulin and induces a G2/M-phase arrest by causing kinetic disruption of microtubule dynamics. Taxol is also able to induce apoptosis through several mechanisms not well described yet inducing activation of gene transcription (e.g. bax, bak), cyclin-dependent kinases, c-jun N-terminal kinase (JNK/SAPK) and phosphorylation of bcl-2 (Srivastava, R. K et al., 1999). Taxol has severe secondary effects due to apoptosis induction in cancer as well as in normal healthy cells.

### DESCRIPTION OF THE INVENTION

The findings of this invention demonstrate that the compounds such as N10-13-10C and N13-13-10C function by modulating the cell cycle and the apoptotic machinery, thus the compounds or their derivatives may be favorably used as agents for prevention and/or therapy of cancers and for the treatment of other proliferative diseases. Moreover, the compounds identified do provide tools to the study of additional molecular targets involved in the induction of the apoptotic process.

The two compounds e.g. N10-13-10C and N13-13-10C derive from the screening of a combinatorial library of trimers of N-alkylglycines were able to induce a G1 arrest and to induce apoptosis.

The N10-13-10C and N13-13-10C compounds posses growth inhibitory properties against a panel of human cancer cell lines representing cancers such human colon adenocarcinoma, human glioblastoma, chronic myelogenous leukemia, human breast cancer and lung cancer. The identified compounds have been identified as inductors of apoptosis as determined by DNA fragmentation in combination with flow cytometry and annexin V assay. Apoptosis is an important cellular function through which chemotherapeutic agents inhibit the growth of cancer cells.

In more detail, N10-13-10C and N13-13-10C, induce G1 cell arrest in exponentially growing cells or in cells synchronized in G0/G1 phase by serum starvation. The G1-arrest in cell cycle progression induced by N13-13-10C was associated with inhibition of pRb and p130 hyperphosphorylation. Moreover, a marked decrease in the E2F dependent protein expression of pRb, p107, cycA, and its activating partner Cdk2 was observed. Finally, an over expression of CKIs, p21^{Cip1} and p27^{kip1}, was shown. The p27^{kip1} levels are thought to be mainly regulated by the ubiquitin-proteosome pathway (Hengst, L. and Reed, S. I., 1996; Shirane, L. et al., 1999). The potential of specific proteasome inhibitors to act as novel-anticancer agents is currently under intensive investigation and therefore, further analyses will be performed to explain the accumulation of p27^{Kip1} and to define the mechanism of action of N10-13-10C and N13-13-10C. p27^{kip1} expression has been reported to be an independent prognostic factor in diagnosis of a broad spectrum of tumors. Reduced or lack of p27^{kip1} expression in human tumors has been associated with high aggressiveness and poor prognosis of various malignant tumors (Lloyd, R. V. et al, 1999; Karter t al. 2000). Ectoptic over expression of p27^{kip1} has associated with failure to induce tumor development in a xenograft model (Chen J. et al., 1996). Thus, N10-13-10C and N13-13-10C are prime candidates for cancer therapy.

Among other goals the initial screen for the selection of compounds took into account to identification compounds that among other effects could synergize the action of Taxol. In the chosen assay it was possible to identify mixtures of compounds which synergize Taxol effect. Some mixtures were found to be inhibitors of cellular proliferation and in combination with Taxol such inhibition was interfered. In this invention we describe the identification of compounds which inhibit cell proliferation, induce G1 cell arrest in exponential cells and in cells synchronized in G0/G1 phase by serum starvation, and are able to induce apoptosis. The compounds have favorable therapeutic profile that qualifies them as anticancer drugs.

The compound induced G1 arrest of cell cycle is observed both, in exponential cells and in G0/G1 synchronized cells and is associated with hypophosphorylation of pRb and p130. Moreover, a marked decrease in the E2F dependent protein expression of pRb, p107, cycA, and its activating partner Cdk2 is observed. Finally, a concomitant induction of p21^{Cip1} and p27^{kip1} is detected. The pro-apoptotic effect of the compounds has been assessed by Annexin V staining and DNA hypodiploidy and has been identified as sub-G1 specific. Another feature of the compounds is that they do not inactivate bcl-xL by phosphorylation.

For screening of the peptoid library containing 10.648 compounds, controlled mixtures of trimers of N-alkylglycine oligomer molecules (peptoid) have been used and constructed under four positional scanning formats. Chemical diversity was introduced through the substitution of position R1, R2 and R3 by 22 different primary amines. 66 controlled mixtures divided into three different subgroups depending on the R1, R2, R3 defined position. The library was screened on a cellular proliferation assay with HT29 human colon adenocarcinoma cells. The compounds were tested either alone or together with a low dose of Taxol (11 nM). After 72 h in culture, cellular viability was measured with the MTT assay.

Some mixtures were found to be inhibitors of cellular proliferation while in combination with Taxol such inhibition was somehow interfered. Dose-response curves were established and 6 mixtures were identified; 4 different amines at R1 position, one amine for R2 position and one for R3 position. Four compounds were then synthesized and called, according to a coded nomenclature, as N4-13-10C, N5-13-10C, N10-13-10C and N13-13-10C (also abbreviated as N4, N5, N10 and N13). They differed from each other at the N-terminal residue. All four compounds inhibited cellular proliferation in the test system, but Taxol prevented the compound's effect only for N10-13-10C (FIG. 1. B) and N13-13-10C (FIG. 1. A). N13-13-10C was the most potent proliferation inhibitor with an IC₅₀=35 μM, followed by N10-13-10C, IC₅₀=40 μM, and then N4-13-10C and N5-13-10C with IC₅₀=100 μM.

When N10-13-10C and N13-13-10C at their IC₅₀ where assayed in combination with serial dilutions of Taxol, a potentated anti-proliferative effect of the compounds was observed versus Taxol alone (FIG. 1. C).

Inhibition of proliferation induced by the compounds was assessed in several cell lines including human colon adenocarcinoma (HT29 and LoVo), human glioblastoma (T98g), chronic myelogenous leukemia (K562), human breast adenocarcinoma (MDA.MB 435 and its lung metastatic derivatives lung 2 and lung 6). The IC₅₀ values for cellular proliferation inhibition (MTT assay) obtained after 72 h treatment with the four compounds are reflected in Table 1.

N10-13-10C and N13-13-10C were able to induce apoptosis in HT29 cells as determined by flow cytometric DNA analysis and sub-G1 peak detection after 72 h treatment. On the contrary, sub-G1 peak was not observed in N4-13-10C and N5-13-10C treated cells (FIG. 2). This observation was not only restricted to HT29 cells. N10-13-10C and N13-13-10C induced ighest apoptosis (50-70%) in HT29 and MDA.MB.435 lung 2 derivative cells (FIG. 3. A). Adenocarcinoma LoVo cells, MDA.MB.435 and its lung 6 derivative showed around 20-30% apoptosis whereas N4-13-10C and N5-13-10C did not.

HT29 cells were treated for 72 h with increasing dose of N10-13-10C or N13-13-10C and subG1 peak was detected by flow cytometry. As shown in FIG. 3. B, N10-13-10C and N13-13-10C treatment in HT29 resulted in a dose-dependent apoptosis.

Time-course analyses were performed to detect the apoptotic features of N10-13-10C and N13-13-10C (FIG. 4. A). Apoptosis was significant in HT29 treated cells already after 48 h and reached a maximum at the highest dose assayed, of 20% for N10-13-10C and around 40% for N13-13-10C at 72 h. Moreover, both compounds alone seemed to increase the percentage of cells in G1-phase after 24 h in culture, whereas no G2/M accumulation was observed with time. However, in combination with low doses of Taxol about 60% of the cell population treated either with N10-13-10C or N13-13-10C was retained in G2/M phase, which was an increased percentage compared with Taxol alone. This would explain the potentated effect of N13-13-10C of the anti-proliferative effect of Taxol on HT29 cells (FIG. 1).

Analysis of DNA staining was performed on HT29 cells treated with N13-13-10C or N10-13-10C for time pulses. Cells were then returned to medium without drugs for up to 72 h. As shown in FIG. 4. B, a minimum of 24 h pulse of N13-13-10C is necessary to induce an irreversible induction of apoptosis. Cell treated for short time pulses of 1, 3 and 6 h with N 13-13-10C show cell cycle profiles that do not differ from control cells.

Early events in apoptosis are the translocation of phospatidyl serine from the inner to the outer leaflet of the plasma membrane which can be monitored via Annexin V, a phospholipid-binding protein with high affinity for phosphatidylserine. Annexin V-FITC detection assay was performed to identify the onset of early apoptosis induced on HT29 cells by N13-13-10C. Time-course analysis of Annexin-V detection showed a 14% of early apoptotic cells (IP negative, Annexin V positive) after 40 h treatment with N13-13-10C (35 μM). This represents a 3.5 fold increase with respect to control cells and is similar to Taxol treated cells (FIG. 5).

As it has been previously reported that JNK mediates intracellular signals for activation of apoptosis in respond to various stressors (Tournier et al., 2000; Xia et al., 1995; Minden A., and Karin, M., 1997; Ip, Y. and Davis, R. J., 1998; Chen et al., 1996; Johnson et al., 1996; Verheij t al., 1996; Park et al., 1997), HT29 cells have been treated with either N10-13-10C or N13-13-10C. The western blot analysis revealed that JNK was activated after 3 to 6 h as observed after incubation of the blots with a JNK-phosphorylation specific antibody (FIG. 6.A).

It is known in the art that anti-apoptotic Bcl-2 proteins prevent cytochrome c release from mitochondria and thereby preserve cell survival. Taxol on the other hand is a microtubule-stabilizing agent and has been described to induce JNK-dependent phosphorylation of Bcl-x_{L} and Bcl-2 (Razandi et al., 2000; Srivastava et al., 1999). Such phosphorylation mediates the inactivation of the anti-apoptotic Bcl-2 protein. A time-course analysis was performed to assess whether JNK activation was related to a phosphorylation of Bcl-x_{L}, a member of the Bcl-2 family of proteins, in HT29 cells treated with N10-13-10C or N13-13-10C alone or in combination with Taxol. A slower migrating band was detected in Taxol-treated HT29 cells extracts which corresponds to phosphorylated bcl-x_{L}, This effect was not observed in N10-13-10C treated cells (FIG. 6. B). Moreover, in combination with Taxol, N10-13-10C treatment did not interfere Taxol induced Bcl-x_{L} hyper-phosphorylation. The same pattern was observed for N13-13-10C treated cells (data not shown). Bax, a pro-apoptotic member of the bcl family was not increased after N10-13-10C and/or Taxol exposure of HT29 cells.

As already mentioned, a slight increase in cells at G0/G1 phase was observed in N10-13-10C or N13-13-10C treated cells after 24 h (FIG. 4. A) and it is expected that this effect is due to an arrest of cells at specific check point(s) in cell cycle. Experimentally this finding was substantiated analysing the compounds in a cellular model with synchronized cells. T98 g cells were arrested in G1 phase (82%) by serum starvation in MCDB 105 medium after 72 h. Upon 10% serum re-addition, cells were allowed to re-enter into the cell cycle. More than 50% of the cells were in the S-phase, and only 20% remained in G1 after 19 h of FCS introduction. Addition of N10-13-10C or N13-13-10C to the cultures at the moment of serum addition restrained S-phase entry (FIG. 7. A). Around 40% of the cells were retained in G1. In conclusions both N10-13-10C and N13-13-10C were able to induce a G1 arrest either in asynchronous or in synchronized cell cultures, as determined by cell cycle DNA analysis. In comparison, Olomucine which is a cdk2 inhibitor, retained 80% of the cells in G1. The topoisomerase II inhibitor etoposide treated cells were arrested in S phase of the cell cycle (85% of the cell population). As Taxol induces a G2/M arrest its action is independent of G1/S checkpoint and it does not effect cell cycle profile after 19 h of treatment.

To confirm the G1 arrest of the cell cycle, we analyzed the DNA synthesis by an BrdU incorporation assay. Serial dilutions of the compounds N4-13-10C, N5-13-10C, N10-13-10C, N13-13-10C, etoposide or olomucine were assayed on synchronized T98 g cells as described in FIG. 7. A. Arrested cells were induced to cell cycle re-entry by re-addition of serum alone or with test compounds for 17 h, followed by 2 h in combination with 10 μM BrdU. As shown in FIGS. 7. B and C, olomucine is a very strong inhibitor of BrdU incorporation (IC₅₀=50 μM), and correlates with the 82% cells in G1 phase observed by DNA staining (FIG. 7. A). The DNA synthesis inhibition ranking is followed by N13-13-10C (IC₅₀=150 μM), N10-13-10C (IC₅₀=100 μM) and etoposide (IC₅₀=200 μM). N5-13-10C inhibits in some extend BrdU incorporation (30% at 180 μM), whereas N4-13-10C does not.

Cell cycle progression maintains its control through several mechanisms and one of them involves checkpoint proteins such as the retinoblastoma pRb. pRb, p130 and p107 and constitutes the so called family of pocket proteins, however after all, only pRb is central in the G1/S checkpoint regulation (Harrington et al., 1998). In its unphosphorylated form pRb binds to and represses E2F, a transcription factor that regulates the transcription of genes which are essential for S-phase progression. After mitogenic stimuli, pRb is partially phosphorylated by cyclin D/cdk4, and releases enough E2F for cyclin E expression. Further, cyclin E/cdk2 completely phosphorylated pRb, releasing free E2F, and promoting E2F-dependent progression to the S-phase.

Upon analysis of the G1-arrest induced by N10-13-10C or N13-13-10C treated cells correlation with the phosphorylation status of pRb has been noticed. Time-course analysis of pRb expression of N10-13-10C and N13-13-10C HT29 treated cells was performed by western blot. We analyzed the specific cycD1/cdk4 phosphorylation of pRb at Ser⁷⁸⁰ (Kitagawa et al., 1996). This pRb phosphorylation at Ser⁷⁸⁰ did not decrease after N13-13-10C treatment of HT29 cells respect to total pRb levels (FIG. 8. A). This result would indicate that cycD1/cdk4 activity is not impaired and that cycE/Cdk2 activity is inhibited in some extend. Moreover, pRb levels decreased in compound-treated cells compared to controls after 24 h in culture.

The effect of N13-13-10C and Taxol on synchronized T98 g cells on protein expression of pocket proteins, cyclin and Cdks involved in G1 check point. As for HT29 cells shown in FIG. 8. A, treatment of T98 g cells with N13-13-10C prevented pRb hyperphosphorylation and decreased total pRb levels (FIG. 8. B). On the other hand, p130 remains also hypophosphorylated, while total levels are increased. Finally, p107 levels are down regulated. The expression of E2F-regulated genes such as cycA, p107 and pRb is down regulated. The time course of pRb phosphorylation correlates well with the G0/G1 arrest by inhibition of cycE/Cdk2 activity. pRb down regulation correlates with apoptosis.

³³Pan Qinase assays containing cycD, Cdk4 and pRb protein in the presence of 3 μM or 30 μM, N10-13-10C or N13-13-10C, were performed. No inhibition of pRb phosphorylation was observed in such assays, confirming that cycD/Cdk4 activity was not affected by N10-13-10C or N13-13-10C (FIG. 8. A).

We had observed that cycA protein levels and pRb hypophosphorylation decrease in cell extracts of N10-13-10C or N13-13-10C treated cells (FIG. 8). To assess whether these compounds were direct inhibitors of Cdk2 activity, in vitro kinase assays for Cdk2 were performed. Neither of both compounds was able to inhibit cycA-Cdk2 kinase activity at 3 μM or 30 μM.

It is known in the art that Cdks are needed for the phosphorylation of Tyr/Thr residues together with the activation by cyclins to promote protein phosphorylation and progression through the cell cycle. Cyc/Cdk activity is negatively regulated by CKI such as p15^{INK4b}, P16^{INK4a}, p21^{Cip1}, and p27^{KIP1} (Sher, C. J. and Roberts, J. M., 1995). We analysed by Westren blot the levels of p21^{Cip1} and p27^{kip1} which are known to regulate the entry of cells at G1/S transition check point (FIG. 8. B). Both, p27^{kip1} and p21^{Cip1} have been described as potentiators of the assembly Cdk4-6/CycD complexes (LaBaer et al., 1997). On the other hand, p27^{kip1} and p21^{Cip1} are potent inhibitors of all Cdk2 complexes, being one molecule of p21^{Cip1} sufficient to completely inhibit their activity (Hengst et al., 1998; Adkins et al., 2000). In normal cells, the amount of p27^{kip1} is high during G0 phase, but it rapidly decreases on reentry into G1/S phases triggered by specific-mitogenic factors, such as TGF□, p53 or AMPc (Poon, R. Y. et al., 1995). Forced expression of p27^{kip1} results in cell arrest in G1 phase (Polyak, K. et al., 1994; Toyoshima, H. and Hunter, T., 1994). Western blot revealed an appreciable induction of p27^{kip1} after 15 h of N13-13-10C treatment in T98 g cells, which was paralleled by the detection of hypophosphorylated pRb.

Analysis of p21^{Cip1}, indicated peak levels after 17 h of N13-13-10C treatment. Over expression of p21^{Cip1} and p27^{Kip1} was also observed in HT29 cells treated with N13-13-10C for 24 h and 48 h (FIG. 8. C). p21^{Cip1} is as well a downstream mediator of p53 (Haapajärvi et al., 1999) and as HT29 cells harbor mutated p53, whereas T98 g cells are wt for p53; it is indicated that induction of p21^{Cip1} expression was p53 independent. Moreover, p53 levels are not altered after N13-13-10C treatment.

### FURTHER EXAMPLES

Synthesis of the library of N-alkylglycines. A library of 10.648 compounds in 66 controlled mixtures was synthesised by using the positional scanning format in solid phase. A collection of 22 commercially available primary amines was used for introducing the desired chemical diversity in the library. The details of this synthesis are described elsewhere (WO0228885). Briefly, starting from Rink amide resin (Rapp Polymere, 0.7 meq.) the eight-step synthetic pathway involved the initial release of the Fmoc protecting group. Then the successive steps of acylation with chloroacetyl chloride followed by the corresponding amination of the chloromethyl intermediate using the particular primary amine or the equimolecular mixture of the 22 amines was conducted as appropriate. All these reactions were carried out in duplicate. Finally the products were released from the resin by using a trifluoroacetic acid-dichloromethane-water mixture, solvents were evaporated and the residues were lyophilised and dissolved in 10% Dimethylsulfoxide (DMSO) at the concentration of 10 mg/ml for screening.

Synthesis of N13-13-10C and N10-13-10C. These compounds were synthesized in a 10 mL polypropylene syringe using as solid suport a polystyrene Rink amide AM RAM resin (0.6 g, load of 0.7 mmol/g, 0.42 mmol). Deprotection: After swelling the resin, a solution containing 5 mL of 20% piperidine in DMF (Dimethylformamide) was added and the mixture was stirred for 30 min at 25° C. The resin was filtered and washed with DMF (3×5 mL), iPrOH (3×5 mL) and DCM (Dichloromethane) (3×5 mL). Acylation: the resin was treated with a solution of chloroacetic acid (198 mg, 2.1 mmol) and N,N′-diisopropylcarbodiimide (2.1 mmol) in 5 mL DCM-DMF (2:1). The reaction mixture was stirred at room temperature for 30 min and filtered. The resin was drained and washed with DCM (3×5 mL), iPrOH (3×5 mL) and DMF (3×5 mL). Amine coupling: a solution of phenethylamine (2.1 mmol) and triethyl amine (2.1 mmol), in 5 ml of DMF, was added to the resin and the suspension was stirred for 3 h at 25° C. The supernatant was removed and the mixture was drained and washed with DMF (3×3 mL), iPrOH (3×3 mL) and CH₂Cl₂ (3×3 mL). The second and third acylation steps and amine couplings were carried out as described above. These two amine couplings were carried out using 4-methoxyphenethylamine (2.1 mmol) in the case of N13-13-10C, and phenethylamine in the third amination step in the case of N10-13-10C. Cleavage: the resin was treated with a mixture of 60:40:2 (v/v/v) TFA/DCM/H₂O for 30 min at 25° C. The cleavage mixture was filtered and joined filtrates were pooled and the solvent removed by evaporation under reduced pressure. All the above processes were carried out in duplicate.

Analytical and Structural Data

Analysis was performed by High performance Liquid chromatography (HPLC) using a Kromasil 100 C8 (15×0.46 cm, 5 μm) column at a flow rate of 1 ml/min. Solvent A consisted of Acetonitrile (CH₃CN) containing 0.07% TFA (Trifluoroacetic acid) and solvent B 0.1% TFA in H₂O. Analytical conditions were established at 2 min 20% solvent A, from 20 to 80% in 17 min and 1 min at 80% solvent A at a flow rate of 1 ml/min and λ 220 nm.

N13-13-10C:

HPLC-MS (ES-APCl): 561.2 (M+1)

¹H-NMR (300 MHz, MeOD-d₄): mixture of conformers at 40° C. 7.7-7.0 (m, 9H, H-arom), 6.9-6.8 (m, 4H, H-arom), 4.3-3.8 (m, 6H, 3×CH₂CO), 3.75-3.73 (s, 6H, CH₃O), 3.6-3.15 (m, 4H, 2×CH₂CH₂N), 3.0 (m, 2H, CH₂NH), 2.9-2.6 (m, 6H, 3×ArCH₂CH₂), 1.3 (t).

¹³C-NMR (300 MHz, MeOD-d₄): mixture of conformers at 40° C.: 173.4 (CO), 170.2, 169.9 (CO), 167.5, 166.9 (CO), 160.4, 159.7 (2×C_{Ar}—CH₃O), 139.8, 139.7 (C_{Ar}), 131.9 (2×C_{Ar}), 131.3-127.4 (9×CH_{Ar}), 115.4, 114.9 (4×CH_{Ar} next to CH₃O), 55.7 (2×CH₃O), 51-48 (3×CH₂CO, 2×CH₂CH₂N), 49.5 (CH₂NH), 35-32 (3×ArCH₂CH₂), 9.1 (CH₂).

N10-13-10C:

HPLC-MS (ES-APCl): 531.2 (M+1)

¹H-NMR (300 MHz, MeOD-d₄): mixture of conformers at 40° C. 7.7-7.0 (m, 12H, H-arom), 6.9-6.8 (m, 2H, H-arom), 4.3-3.8 (m, 6H; 3×CH₂CO), 3.7 (s, 3H, CH₃O), 3.6-3.15 (m, 4H, 2×CH₂CH₂N), 3.0 (m, 2H, CH₂NH), 2.9-2.6 (m, 6H, 3×ArCH₂CH₂), 1.3 (t).

¹³C-NMR (300 MHz, MeOD-d₄): mixture of conformers at 40° C.: 173.9, 173.1 (CO), 170.2, 169.9 (CO), 167.5, 166.9 (CO), 160.1, 159.7 (C_{Ar}—CH₃O), 139.8, 139.7 (C_{Ar}), 137.6, 137.5 (C_{Ar}), 131.9 (C_{Ar}), 131.3-127.4 (12×CH_{Ar}), 115.2, 114.9 (2×CH_{Ar} next to CH₃O), 55.6 (CH₃O), 51-48 (3×CH₂CO, 2×CH₂CH₂N), 49.5 (CH₂NH), 35-32 (3×ArCH₂CH₂), 9.1 (CH₂).

Cells. HT29 and LoVo (human colon adenocarcinoma) and MDA.MB.435 cells and their derivatives MDA.MB.435 Lung2 and MDA.MB.435 Lung6 (human breast adenocarcinoma) cells were cultured in DMEM-F12 medium containing 10% of fetal calf serum (FCS). Human glioblastoma T98 g and chronic myelogenous leukemia K562 cells were grown in RPMI 1640 medium containing 10% FCS. All cells were used in their exponential growth phase and tested to be Mycoplasma free with EZ-PCR Mycoplasma test kit (Biological Industries).

Cellular Assays. Combinatorial libraries were screened on a cellular proliferation assay with HT29 human colon adenocarcinoma cells. The compounds were tested either alone or together with low doses of Taxol (nM). After 3 days in culture, cellular viability was measured with an MTT assay (3-4,5-Dimethyl-2-thiazolyl-2,5-diphenyl-2H-tetrazolium bromide). MTT was added at a final concentration of 1 mg/ml in the cell cultures, and after 4 h incubation at 37° C. cell lysis was performed with 15% SDS/DMF (v/v). Spectrophotometric measurement of MTT-formazan at 570 nm and a reference filter at 630 nm allows quantitation of cellular viability. Proliferation inhibition due to the compounds alone was compared to inhibition observed in Taxol plus compound treated cultures.

Annexin V assay. Treated cells were harvested with EDTA 0.02% in Hank's Balanced Salt Solution (HBSS), washed in HBSS then in PBS (phosphate buffered saline) containing 1% BSA (Bovine serum albumine) and finally resuspended in Annexin V incubation buffer (10 mM HEPES 7.4; 140 mM NaCl; 2.5 mM CaCl₂) containing 1% BSA. 10⁵ cells were incubated with 5 μl Annexin-V-FITC (Bender MedSystems) for 1 h at room temperature and in the dark. Dead cells were stained with Propidium Iodide (PI) at 2 μg/ml. The analysis was immediately performed by flow cytometry.

DNA analysis. Floating and adherent cells treated with the compounds were collected by trypsinization and washed twice with PBS. The cells were permeabilized overnight at −20° C. with ice-cold ethanol 70%. The cells were washed in PBS, adjusted at 0.5×10⁶ cells/ml and incubated with 20 μg/ml Propidium Iodide and 2 μl/ml RNase DNase-free for 30 min at 30° C. Cells were maintained overnight at 4° C. and then analysed by flow cytometry. Flow cytometric experiments were carried out using an Epics XL flow cytometer (Coulter Corporation, Hialeah, Fla.). The instrumet was set up with the standard configuration: excitation of the sample was done using an standard 488 nm air-cooled argon-ion laser at 15 mW power. Forward scatter (FSC), side scatter (SSC) and red (620 nm) fluorescence for PI were adquired. Optical alignement was based on optimized signal from 10 nm fluorescent beads (Immunocheck, Epics Division). Time was used as a control of the stability of the instrument. Red fluorescence was projected on a 1024 monoparametrical histogram. Aggregates were excluded gating single cells by their area vs. peak fluorescence signal. DNA analysis (Ploidy analysis) on single fluorescence histograms was done using Multicycle software (Phoenix Flow Systems, San Diego, Calif.).

Western blotting. Floating and adherent cells were harvested and pellets were resuspended in RIPA buffer (50 mM Tris/HCl 7.4, 250 mM NaCl, 0,5% Igepal CA630, 5 mM EDTA, 1 mM PMSF, 10 μg/ml leupeptin, 50 mM NaF, 0.1 mM Na₃VO₄) or Deoxycholate buffer (10 mM phosphate buffer 7.4, 0.1 mM NaCl, 0.5% Deoxycholate, 1% Igepal, 0.1% SDS, 1 mM PMSF). Protein concentration was determined with BCA Protein Assay Kit (Pierce) for RIPA extracts or Bradford assay (BioRad) for Deoxycholate extracts. Total protein (20-30 μg/lane) were separated by SDS-PAGE, transferred to PVDF membranes (Gellman), and probed with antibodies against Bcl-x_{L} (Transduction); Bax (Santa Cruz); JNK (Santa Cruz); phospho-JNK (Cell Signaling); pRb (Pharmingen); pRb-phospho Ser780 (Cell Signalling); p130 (Santa Cruz); p107 (Santa Cruz); Cdk2 (Santa Cruz); p27^{Kip1} (Santa Cruz); p21^{Cip1} (Santa Cruz); actin (Sigma); or tubulin (ICN) and developed with ECL system (AmershamPharmacia biotech)

BrdU assay. T98g glioblastoma cells at 5000 cells/well in microtiter plates were arrested in G1-phase for 72 h by serum deprivation in MCDB 105 medium. By serum (10%) readdition, cells were treated with serial dilutions of the compounds for 17 h, and followed in combination 10 μM BrdU for 2½ h. BrdU incorporation, i.e. DNA synthesis, was quantified with Cell Proliferation ELISA system, vs. 2 (AmershamPharmacia biotech) as described by the manufacturer.

Kinase assays for testing of Cdk2/CycA-E kinase activity. ELISA plates were blocked with 200 μl of blocking solution (PBS containing 1% BSA, 0.02% Tween and 0.02% sodium Azide) overnight at 4° C. Plates were then subsequently washed 3 times, 5 min each with 100 μl of washing solution (PBS containing 0.02% Tween and 0.02% sodium Azide). Plates were then dried during 2-4 h at room temperature. Kinase assay was performed in kinase buffer (Hepes 25 mM pH 7.4 and MgCl₂ 10 mM) containing 4 μg of histone H₁, 30 μM ATP, 2 mM DTT, 0.1 μl of ATP-P³², 800 nM GST-CDK2, and 800 nM of GST-cyclin A in a final volume of 60 μl. Assays were carried out in the presence or absence of different concentrations of peptide mixtures to be checked. A inhibitory control was performed adding 800 nM of p21 to the reaction media. Mixtures were incubated for 30 min at 37° C. After incubation, 50 μl of each mixture was filtered in nitrocellulose membranes placed in a dot blot apparatus. Then, samples were washed with 200 μl of kinase buffer, then with 35 μl de TCA 10%, and finally with two washes of 100 μl TCA 10% followed by 100 μl H₂O. After this process, membranes were dried at room temperature. The radioactivity associated to the membranes was detected with a “Phosphor-imager”.

To assay Cdk4/CycD1 kinase activity, Cdk4 was expressed in Sf9 insect cells as recombinant GST-fusion protein by means of baculovirus expression system. Kinase assay was performed in 96-well FlashPlates (NEN) in a 50 μl reaction volume using the ³³PanQinase activity assay (ProQinase) and Beckman Coulter/Sagian robotic system. The reaction cocktail was 20 μl of assay buffer (50 mM Hepes-NaOH pH 7.5, 3 mM MgCl₂, 3 mM MnCl₂, 3 μM Na-orthovanadate, 1 mM DTT, 0.1 μM (³³P)-dATP); 1 μg pRb protein; 100 ng enzyme; and 5 μl of test compound in 10% DMSO. The reaction cocktail was incubated at 30° C. for 80 min. The reaction was stopped with 50 μl of 0.2% (v/v) H₃PO₄, plates were aspirated and washed two times with 0.9% (w/v) NaCl. Incorporation of ³³P was determined with a microplate scintillation counter (Microbeta, Wallac).

### FIGURE LEGENDS

FIG. 1. Compounds N10-13-10C, N13-13-10C, N4-13-10C and N5-13-10C inhibit HT29 proliferation. Taxol interferes the effect of N10-13-10C and N13-13-10C. (A, B) HT29 cells were grown with peptoid at several concentrations with or without Taxol (11 nM). MTT assay was performed after 72 h of treatment. Proliferation inhibition is shown for N4-13-10C and N13-13-10C (A.), N5-13-10C and N10-13-10C (B.) compared to proliferation of control cells. IC₅₀ value is specified for each peptoid. N10-13-10C and N13-13-10C were purified peptoids by HPLC, corresponding to the major and active fraction. N4-13-10C and N5-13-10C peptoids were not purified by HPLC. (C) HT29 cells were treated with serial dilutions of Taxol alone or combined with IC₅₀ value of N13-13-10C (35μM) or N10-13-10C (40 μM). Proliferation was evaluated by MTT assay after 72 h of treatment. An arbitrary value of 100% was assigned to the densiometric rate of untreated cultures and all other values are depicted relative to that reference. The values are means of six (n=6) replicates.

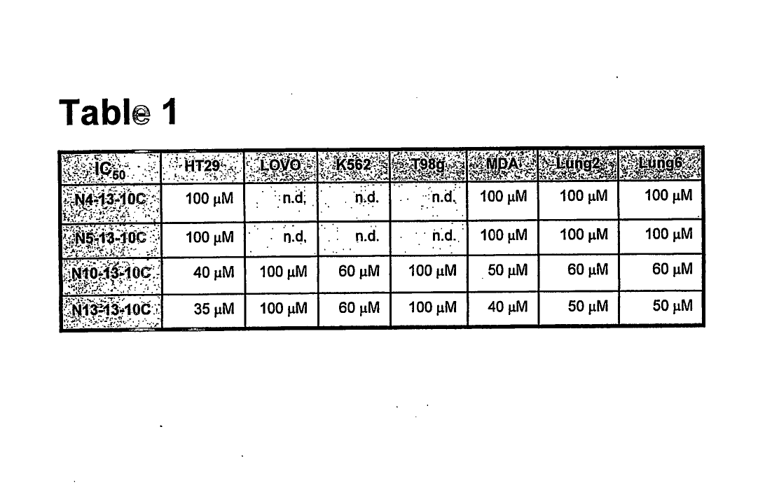

FIG. 2. N10-13-10C and N13-13-10C are pro-apoptotic peptoids while N4-13-10C and N5-13-10C are not. Cell cycle profile was analysed by DNA staining. HT29 cells were grown for 72 h in presence of N4-13-10C (100 μM), N5-13-10C (100 μM), N10-13-10C (40 μM), N13-13-10C (35 μM), Taxol (11 nM) or DMSO as negative control. Fraction of cells at G0/G1, S, G2/M or subG1 peak are specified.

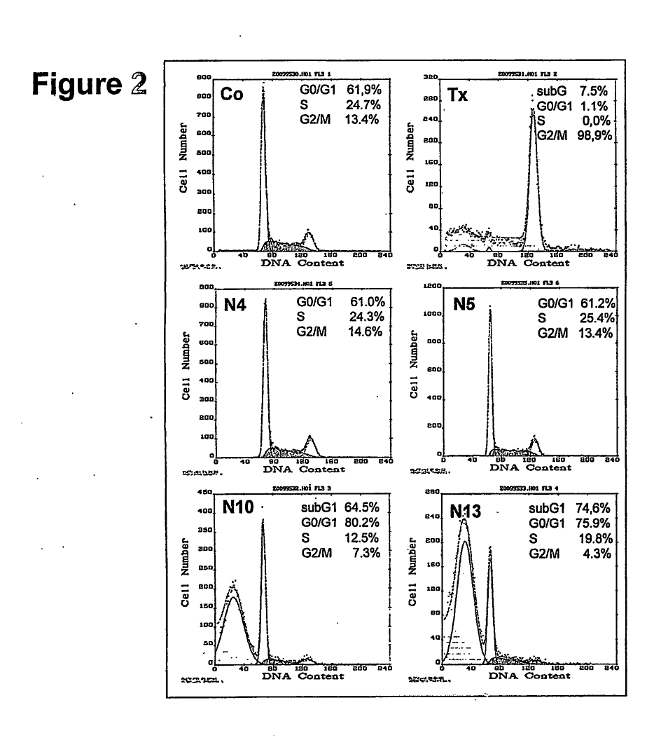

FIG. 3. Specific pro apoptotic effect of N10-13-10C and N13-13-10C. (A) SubG1 peak analysis on several cell lines including HT29, LoVo, MDA.MB.435 and its lung metastatic derivatives lung 2 and lung 6 after 72 h treatment with N10-13-10C or N13-13-10C at IC₅₀ values specified on Table 1. (B) Dose-response analysis of subG1 peak after 72 h treatment of HT29 cells with N 10-13-10C or N13-13-10C at 1, 5, 10, 20, 30 and 35 or 40 μM, respectively. Both, floating and adherent cells were collected, fixed, stained with propidium iodide and DNA content was evaluated by flow cytometry. The fraction of cells with hypodiplod DNA content, i.e. subG1 peak are shown.

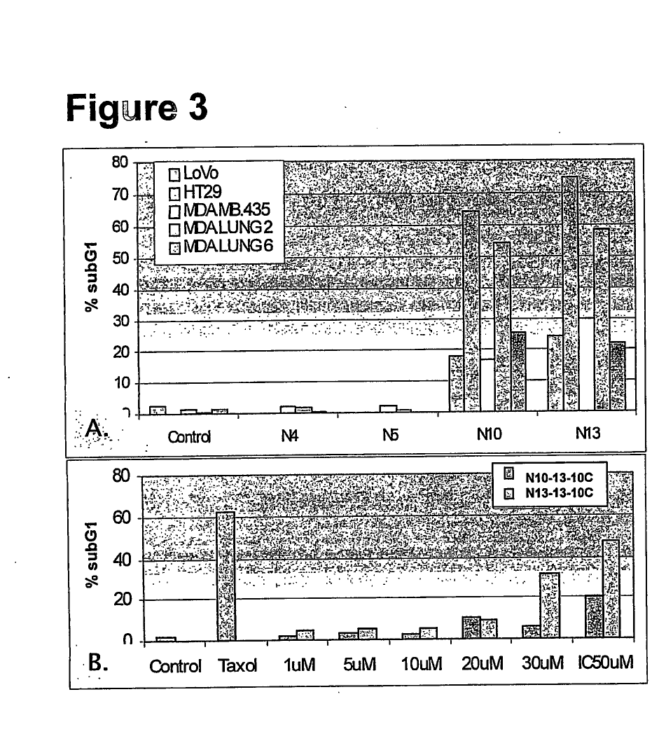

FIG. 4. (A) Time course analysis of cell cycle profile after treatment of HT29 cells with N10-13-10C (40 μM), N13-13-10C (35 μM) and/or Taxol (11 nM) for 24 h, 48 h and 72 h. The fraction of cells with subG1 (dark blue), G0/G1 (red), S (yellow), G2/M (blue) DNA content are shown as % of total cell population. (B.) N13-13-10C pulse experiments. HT29 cells were treated transiently with 35 μM N13-13-10C for 1, 3, 6, 24, 48 or 72 h and returned to medium without product for up to 72 h. DNA staining was analyzed by flow cytometry.

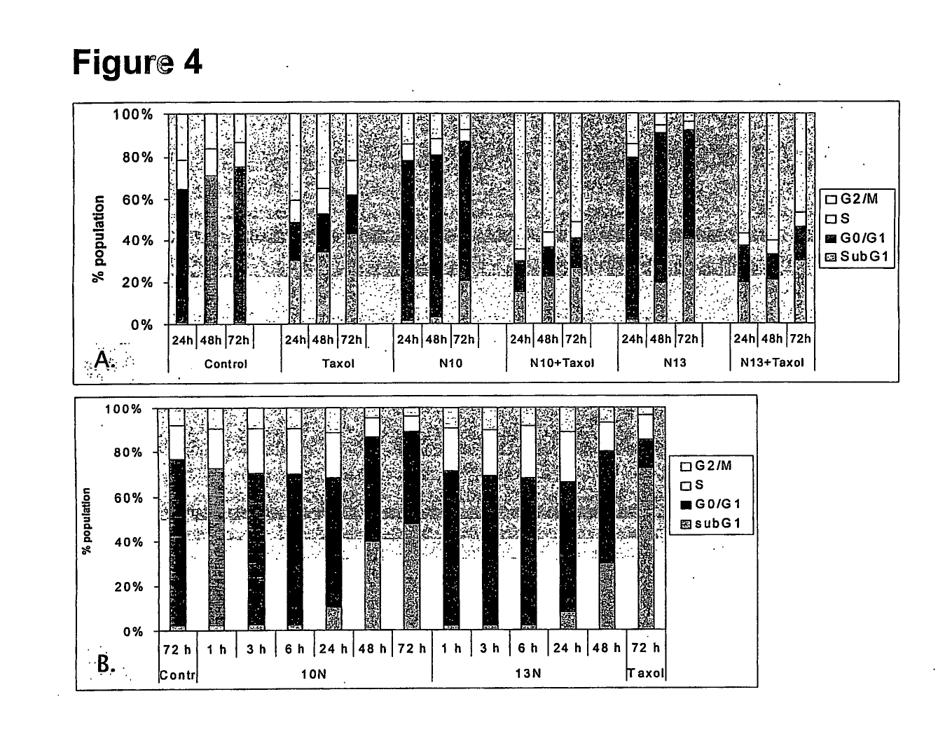

FIG. 5. Detection of early apoptosis. Annexin V assay after 40 h of treatment with DMSO (right panel), N13-13-10C, 35 □M (central panel), or Taxol, 11 nM (left panel). HT29 treated cells were stained with Annexin V-FITC and PI and subjected to flow cytometry. Fluorescence dot blots of annexin V positive (vertical axis) and PI positive (horizontal axis, logarithmic values) cells are shown. Cell distribution expressed as percentage of the population is indicated: early apoptotic cells in quadrant 1, Q1 (AnV+/IP−); dead cells in Q2 (AnV+/IP+); living cells in Q3 (AnV−/IP−); necrotic cells in Q4 (AnV−/IP+).

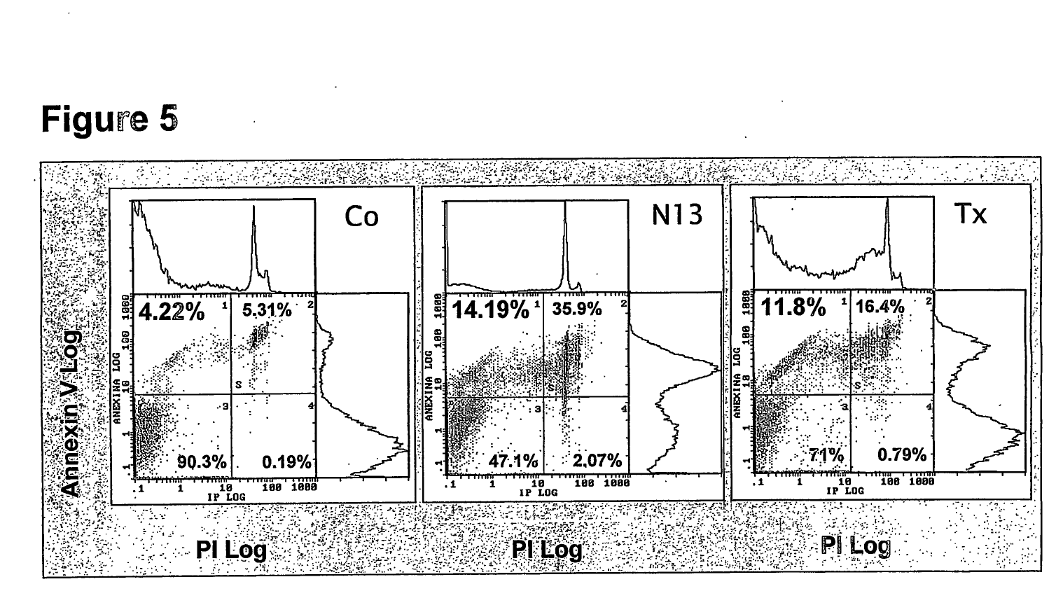

FIG. 6. (A) JNK is activated after N13-13-10C treatment. HT29 cells were treated for 1, 3, 6 and 24 h with N13-13-10C (35 μM), harvested and immunoblotted to assess activation of SAP/JNK MAP kinase. Western blot was first probed with the JNK-phosphor specific antibody, was stripped and reprobed with the pan-JNK antibody and actin to normalize total protein level. Phosphorylation of JNK was observed at 3-6 h after N13-13-10C treatment. (B) Bcl-xL is not posttranslationally modified by phosphorylation after N10-13-10C treatment. HT 29 cells were treated with N10-13-10C (40 μM), Taxol (11 nM) or combination of both for 3, 6 and 24 h. Extracts from floating and adherent cells were immunoblotted against Bcl-xL, Bax and actin to normalize to total protein loading.

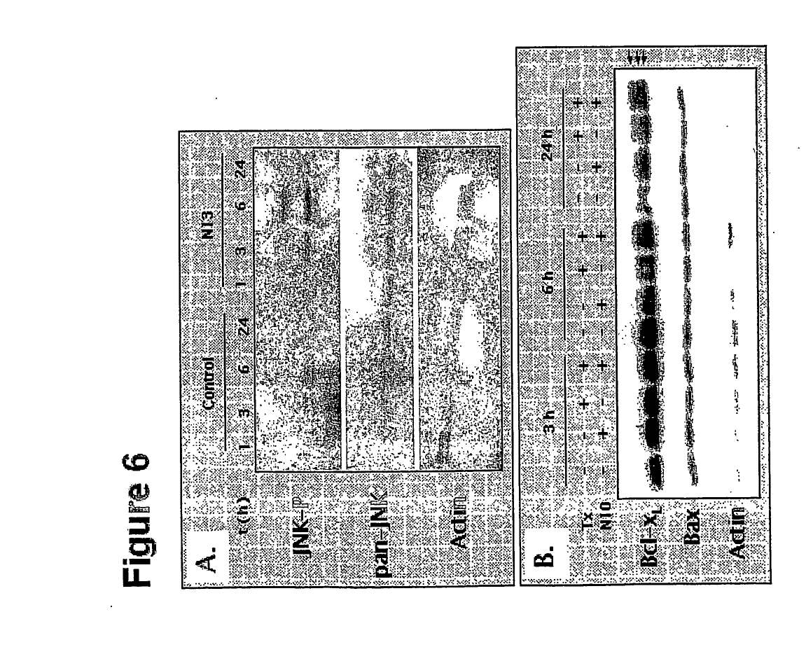

FIG. 7. N10-13-10C and N13-13-10C induce a G1 cell cycle arrest while N4-13-10C and N5-13-10C do not. (A) T98 g cells were synchronized and reinitiated for 19 h with FCS alone or in combination with N10-13-10C (100 μM), N13-13-10C (100 μM), Olomucine (100 μM), etoposide (5 μM) or Taxol (30 nM). Cell cycle profile was assessed by propidium iodide DNA staining and analyzed by flow cytometry. The fraction of cells with G1, S, G2/M DNA content are shown as % of total cell population. (B. and C.) Synchronized T98 g cells were reinitiated with FCS alone or in combination with serial dilutions of N4-13-10C, N5-13-10C, N10-13-10C, N13-13-10C, Olomucine or etoposide for 19 h and labeled with BrdU during the last 2 h. BrdU incorporation was assessed by ELISA using anti-BrdU antibodies. An arbitrary value of 100% was assigned to the densiometric rate of DNA synthesis by untreated cultures and all other values are depicted relative to that reference. The values are means of three (n=3) replicates.

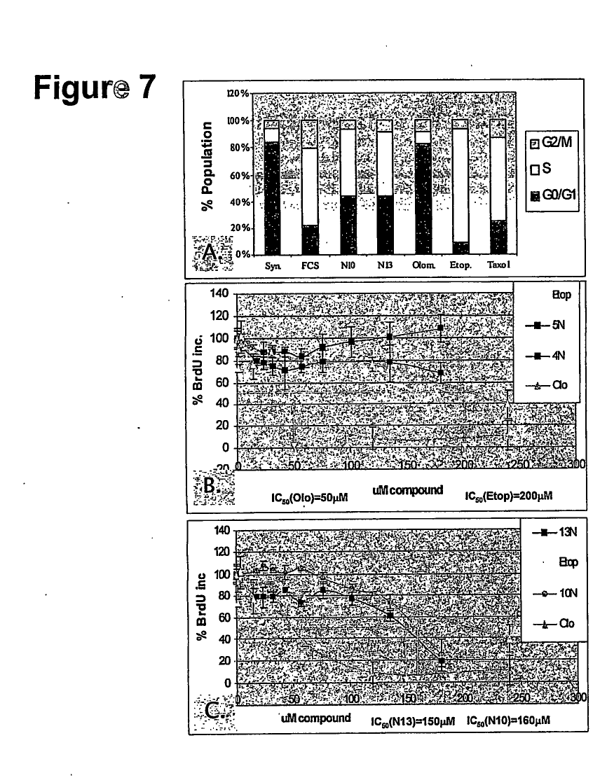

FIG. 8. Expression of protein involved in G0/G1 checkpoint. (A.) HT29 cells were treated for 1, 3, 6 and 24 h with N10-13-10C (40 μM) or N13-13-10C (35 □M), harvested and immunoblotted to assess total pRb levels or Cdk4 specific phosphorylation at pRb-Ser⁷⁸⁰ (PpRb). Western blot was first probed with the pRb-Ser⁷⁸⁰ phosphorylation specific antibody, was stripped and reprobed with the pan-pRb antibody and tubulin to normalize total protein level. (B.) T98 g cells were synchronized by serum starvation and reinitiated with 10% FCS and DMSO (C), N13-13-10C (100 μM) or Taxol (30 nM) for 15, 17, 24 or 29 h. Western blot of total protein extracts was probed with antibodies to p130, pRb, p107, Cdk2, cycA, p27, p21 and actin. Unphosphorylated, hypophosphorylated as well as hyperphosphorylated pRb are detected (arrows). (C.) HT29 cells treated for 24 and 48 h with N13-13-10C (40 μM) or DMSO as control. Immunoblots were stained with antibodies to Cdk2, cycA, p21, p27 and actin.

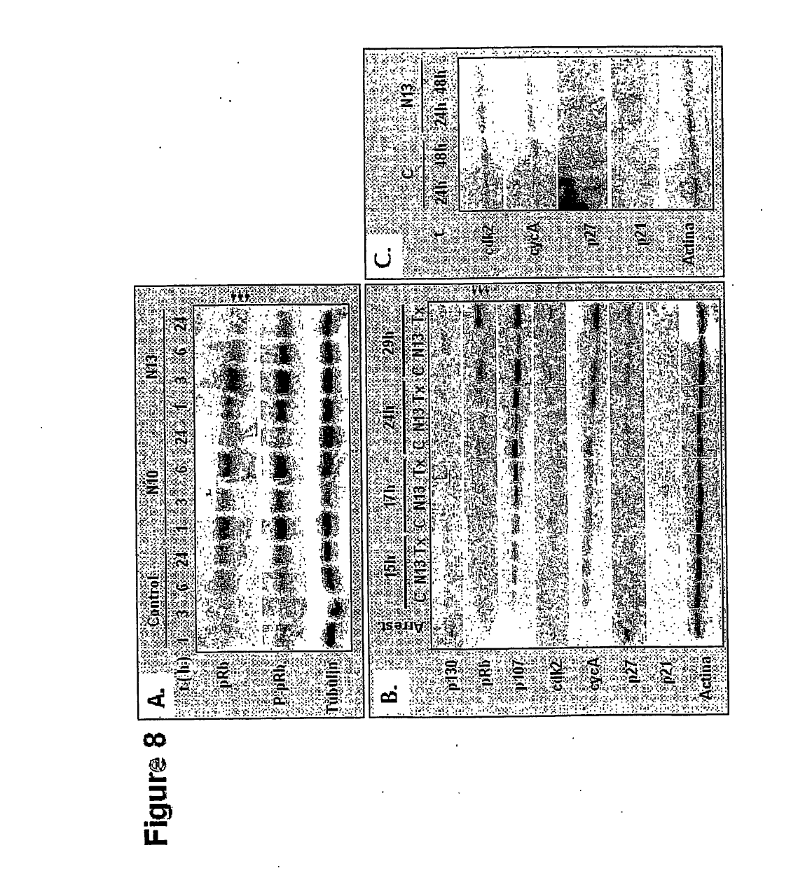

Table 1. N10-13-10C and N13-13-10C posses growth inhibitory properties against a panel of human cancer cell lines including HT29, LoVo, K562, T98g, MDA.MB.435 and its lung metastatic derivatives lung2 and lung6. For each cell line IC₅₀ for N10-13-10C, N13-13-10C, N4-13-10C and N5-13-10C was determined (unless specified as n.d.) by MTT assay after 72 h of treatment.

### CITED LITERATURE

Adkins, J. N. and Lumb, K. J. (2000) Stoichemestry of cyclin A-cyclin-dependent kinase 2 inhibition by p21cip1/Waf1. Biochemistry 39:13925-13930

Chellappan, s. P., et al., (1991) The E2F transcription factor is a cellular target for the RB protein. Cell 65:1053-61

Chen, J. et al. (1996) Tumor suppression and inhibition of aneuploid cell accumulation in human brain tumor cells by ectopic overexpression of the cyclin-dependent kinase inhibitor p27kip1. J. Clin Invest. 97:1983-1988

Chen, Y. R. et al. (1996) The role of c-Jun N-terminal kinase (JNK) in apoptosis induced by ultraviolet C and gamma radiation. Duration of JNK activation may determine cell death and proliferation. J. Biol Chem 271(50):31929-31936

Flores-Rozas, H. et al. (1994) Cdk-interacting protein 1 directly binds with proliferating cell nuclear antigen and inhibits DNA replication catalyzed by the DNA polymerase delta holoenzyme. Proc. Natl. Acad. Sci. USA 91:8655-8659

Haapajärvi, T. et al. (1999) UV radiation is a transcriptional inducer of p21(Cip1/Waf1) cyclin-kinase inhibitor in a p53-independent manner. Exp Cell Res. 248(1):272-9

Hall, M. and Peters, G. (1996) Genetic alterations of cyclins, cyclin dependent kinases, and cdk inhibitors in human cancer. Adv. Cancer Res. 68:67-108

Harrington, E. A. et al., (1998) pRb plays an essential role in cell cycle arrest induced by DNA damage. Proc. Natl. Acad. Sci. USA 95:11945-50

Hengst, L. et al. (1998) Complete inhibition of Cdk/cyclin by one molecule of p21Cip1. Genes Dev. 12:3882-3888

Hengst, L. and Reed, S. I. (1996) Translational control of p27Kip1 accumulation during the cell cycle. Science 271(5257):1861-84

Huse, M. and Kuriyan, J. (2002) The conformational plasticity of protein kinases. Cell 109:275-282

Ip, Y. T. and Davis, R. J. (1998) Signal transduction by the c-Jun N-terminal kinase (JNK)-from inflammation to development. Curr. Opin. Cell Bol. 10(2):205-219

Johnson, N. L., et al. (1996) Signal transduction pathways Regulated by Mitogen-activated/Extracellular response kinase kinase kinase induce cell Death J. Biol Chem 271:3229-3237

Katner, A L. et al. (2002) A recombinant Adenovirus expressing p27(kip1) induces cell cycle arrest and apoptosis in human 786-0 renal carcinome cells. J Urol 168(2):766-773

Katayose, Y. et al. (1997) Promoting apoptsis: a novel activity associated with the cyclin-dependent kinase inhibitor p27. Cancer Res 57:5441-5445

Kitagawa, M. et al. (1996) The consensus motif for phosphorylation by cyclinD1-Cdk4 is different from that for phosphorylation by cyclin A/E-Cdk2. EMBO J. 15(24):7060-9

LaBaer, J. et al. (1997) New functional activities for the p21 family of CDK inhibitors. Genes and Dev. 11:847-862

Levkau, B. et al. (1998) Cleavge of p21^{Cip1/Waf1} and p27^{kip}1 mediates apoptosis in endothelial cells through activation of Cdk2: role of a caspase casacade. Mol Cell 1:553-563

Minden A., and Karin, M. (1997) Regulation and function of the JNK subgroup of MAP kinases. Biochem. Biophys. Acta 1333(2): F85-104

Park, J. et al., (1997) Activation of c-Jun N-terminal Kinase Antagonizes an Anti-apoptotic Action of Bcl-2. J. Biol Chem 272:16725-16728

Poon, R. Y. et al. (1995) Redistribution pf the CDK inhibitor p27 between different cyclin. CDK complexes in the mouse fibroblast cell cycle and in cells arrested with lovastatin or ultraviolet irradiation. Mol Biol Cell 6:1197-1213

Polyak, K. et al. (1994) Cloning of p27Kip1, a cyclin-dependent kinase inhibitor and a potential mediator of extracellular antimitogenic signals Cell 78(1):59-66

Razandi, M. et al. (2000) Plasma membrane estrogen receptors signal to antiapoptosis in breast cancer. Mol. Endo 14(9): 1434-1447

Sherr, C. J. and Roberts, J. M. (1995) Inhibitors of mammalian G1 cyclin-dependent kinases. Genes Dev. 9:1149-63

Shirane, M. et al. (1999) Down-regulation of p27^{Kip1} by Two Mechanisms, Ubiquitin-mediated Degradation and Proteolytic Processing. J Biol Chem 274:13886-13893

Srivastava, R. K. et al. (1999) Deletion of the loop region of Bcl-2 completely blocks paclitaxel-induced apoptosis. Proc. Natl. Acad. Sci. USA 96:3775-3780

Tournier, et al. (2000) Requirement of JNK for stress-induced activation of the cytochrome c-mediated death pathway. Science. 288(5457):870-4

Toyoshima, H. and Hunter, T. (1994) p27, a novel inhibitor of G1 cyclin-Cdk protein kinase activity, is related to p21. Cell 78(1):67-74

Verheij, et al., (1996) Requirement for ceramide-initiated SAPK/JNK signalling in stress-induced apoptosis. Nature 380(6569):75-79

Xia, et al. (1995) Opposing effects of ERK and JNK-p38 MAP kinases on apoptosis. Science. 270:1326-1331

1. A pharmaceutical composition comprising Taxol and a compound of formula I, 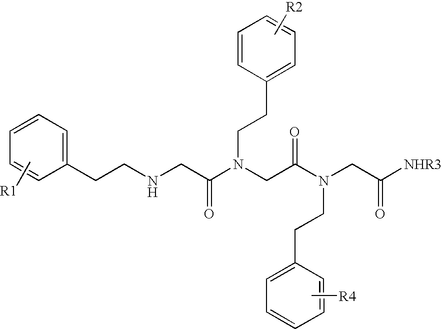
wherein:
R′ is H or OR′
R² is H, R″
R³ is H, R″
R⁴ is H, R″, OR′
R′ is C₁-C₆ alkyl residue
R″ is methyl, ethyl, butyl

2. The composition according to claim 1, wherein R¹ is H, R² is OR′, R³ is H and OR′ is in para-position.

3. The composition according to claim 1, wherein R¹ is OR′, R² is OR′, R⁴ is H and OR′ is in para-position.

4. The composition according to claim 1, wherein R⁴ is H.

5. Use of a composition according to claim 1 for the preparation of a medicament for induction of apoptosis.

6. Use of a composition according to claim 1 for the preparation of a medicament for the treatment of hyperproliferative disease.

7. Use of a composition according to claim 1, wherein the compounds are applied concurrently or sequentially

8. A pharmaceutical kit of parts comprising a first package with Taxol and a second package comprising a compound of formula I.

9. A method of cancer therapy which comprises:
performing a first treatment with Taxol; and performing on the patient a second treatment with a compound according to claim 1 which induces apoptosis in said cancer cells, wherein the first and second treatment are performed in any order or concurrently
